(19) United States
(12) Patent Application Publication
(10) Pub. No.: US 2014/0249077 A1
(43) Pub. Date: Sep. 4, 2014

(54) GEL COMPOSITIONS

(71) Applicants: ASTRAZENECA PHARMACEUTICALS LP, WILMINGTON, DE (US); AMYLIN PHARMACEUTICALS, SAN DIEGO, CA (US)

(72) Inventors: Andrew Xian Chen, San Diego, CA (US); Hailiang Chen, San Diego, CA (US)

(73) Assignees: ASTRAZENECA PHARMACEUTICALS LP, WILMINGTON, DE (US); AMYLIN PHARMACEUTICALS, LLC, SAN DIEGO, CA (US)

(21) Appl. No.: 14/122,796

(22) PCT Filed: Jun. 8, 2012
(86) PCT No.: PCT/US12/41519

§ 371 Date: Mar. 17, 2014

Related U.S. Application Data
(60) Provisional application No. 61/495,282, filed on Jun. 9, 2011.
(60) Provisional application No. 61/500,042, filed on Jun. 22, 2011.

(51) Int. Cl. A61K 38/22; A61K 47/24; A61K 9/00

(52) U.S. Cl. CPC A61K 38/2264; A61K 38/22; A61K 9/0024; A61K 47/24
USPC 514/5.3; 514/9.7; 514/5.8; 514/7.3; 514/6.9

(57) ABSTRACT

The present invention is directed to compositions and methods of preparation of phospholipid gels.

### SEQUENCE LISTING

The instant application contains a Sequence Listing which has been submitted in ASCII format via EFS-Web and is hereby incorporated by reference in its entirety. Said ASCII copy, created on May 18, 2012, is named 2202USPRO.txt and is 2,024 bytes in size.

### FIELD OF THE INVENTION

The present invention relates to gel compositions comprising at least one active pharmaceutical ingredient selected from pramlintide, a pramlintide analog, metreleptin, and a metreleptin analog (API) and at least one phospholipid. Preferably, the gel compositions allow for sustained delivery of at least one API by no more than once daily injection.

### BACKGROUND OF THE INVENTION

Leptin is a neurohormone that is predominantly secreted by adipocytes and binds to receptors in the hypothalamus. Leptin plays a key role in regulating long-term energy homeostasis. Leptin-deficient humans exhibit severe hyperphagia and profound obesity, which can be reversed by leptin replacement (Nature. 1997 Jun. 26; 387 (6636):903-8). Recombinant human methionyl leptin, also known as metreleptin, has been studied as a potential treatment for obesity, type 2 diabetes, and lipodystrophy.

Amylin is a peptide hormone co-secreted with insulin by pancreatic beta cells after nutrient ingestion whose primary physiological roles involve the inhibition of feeding behavior and gastric emptying, and subsequently reduced body weight, as well as lowering meal-related blood glucose levels. Secreted from pancreatic beta cells in response to meals, its overall effect is to slow the rate of appearance (Ra) from the meal, which is mediated via a coordinate reduction of food intake, slowing of gastric emptying, and inhibition of digestive secretion (gastric acid, pancreatic enzymes, and bile ejection). An amylin analog, pramlintide, has been shown in several clinical trials to increase satiation, reduce food intake, and elicit durable weight loss in obese individuals (Am J Physiol Endocrinol Metab. 2007 August; 293(2):E620-7. Epub 2007 May 15).

Human clinical studies have shown that a combination of metreleptin, and pramlintide is effective in reducing weight in obese patients following subcutaneous injections, making this combination a promising weight-loss drug combination (Obesity (Silver Spring). 2009 September; 17(9): 1736-1743)

In a solution formulation, both metreleptin and pramlintide have short elimination half-lives following a subcutaneous injection. For example, following a subcutaneous injection in rats, the plasma concentration of metreleptin drops quickly to less than 1 ng/mL and pramlintide to less than 1 picogram/mL within about 12 hours (FIG. 3), thus requiring frequent injections, e.g., 2 to 3 injections per day, in order to maintain their concentrations in blood at the efficacious levels. A formulation capable of sustained delivery of metreleptin and/or pramlintide and thus allow for no more than once daily injection is desired.

This invention relates to gel compositions comprising at least one active pharmaceutical ingredient selected from pramlintide, a pramlintide analog, metreleptin, and a metreleptin analog (API) and at least one phospholipid, that are capable of forming a depot at the subcutaneous injection site and subsequently prolong the action of the active agent(s) by releasing it into surrounding tissues from the depot reservoir slowly over time. A release profile of no less than daily, such as a 1-day release profile or up to a 7-day depot release profile, which enables a once-a-day or up to once-a-week injection schedule, respectively, would be highly desirable for convenience and better patient compliance.

Phospholipids are naturally occurring substances in the human body and are the major constituents of cell membranes. These molecules have an established record of safety and biocompatibility as components in injected medicines. Phospholipids are also generally insoluble in water or aqueous body fluids. Upon injection into a tissue, phospholipids can precipitate and trap a co-administered drug to form a drug-phospholipid co-precipitate that can function as a depot. Over time, this mass erodes and diffuses slowly into a surrounding tissue and/or is degraded by phospholipase, which is an enzyme distributed throughout the body that slowly hydrolyzes phospholipids, resulting in a slow release of the trapped drug. With such favorable safety, solubility and biocompatibility properties, it would appear that phospholipids are ideal depot materials. However, to date, there have been few successful depot drug products based on phospholipids. One primary problem is the poor injectability associated with phospholipid-based compositions.

The inventors have discovered that a high concentration (i.e., 20-40%) of phospholipids is generally required in order to form the mass that permits depot functionality. However, once the phospholipid concentration exceeds about 20% in a composition, the composition becomes thick, viscous and difficult to inject through fine needles without using an excessively high force. For example, Phosal 50PG, Phosal 50SA, and Phosal 50MCT (produced by the America Lecithin Company) are liposome-forming compositions containing about 50% phospholipids dissolved in propylene glycol/ethanol, oil, and medium chain oil, respectively. With their honey-like consistency, the Phosal compositions are very difficult to inject using a conventional hypodermic needle and syringe. It requires more than 90 Newtons (equivalent to about 20 force pounds) of force to extrude Phosal through a 25G ½ inch long needle from a 1 cc syringe at a plunger speed of 2 cc/min. Thus, it will take 2-5 minutes or more to manually extrude 1 mL of the Phosal-based depot through a 26G needle even using a very high force—which is impractical for general medical use and definitely not suitable for self-administration. Therefore, acceptable injectability using fine hyperdermic needles has been a main hurdle preventing phospholipids from becoming useful depot materials. This invention discloses phospholipid gel compositions with surprisingly good injectability that meets the Acceptable Injectability Criterion, as defined herein.

Another difficulty working with phospholipids is that phospholipids are only soluble in certain organic solvents (e.g., ethanol) or oil (e.g., vegetable oil) while APIs such as metreleptin or pramlintide are only soluble in water, but not in the solvents or oils that can dissolve phospholipids. Furthermore, as proteins, some APIs, such as metreleptin, are damaged quickly upon contact with an organic solvent like ethanol. Therefore, it has been impossible to manufacture a phospholipid-based gel containing a protein drug by the conventional solvent methods or other methods disclosed in the prior art without having the solvent-sensitive drugs precipitate or degrade (See WO 2006/002050, U.S. Pat. No. 5,807,573, WO/1994/008623, U.S. Pat. No. 5,004,611 and Harry Tiemesseen, et al. (2004) European Journal of Pharmaceutics and Biopharmaceutics Volume 58 (2005), pp 587-593). This invention teaches a method which uses an oil-in-water emulsion (instead of a solvent that can damage the APIs) to dissolve both the phospholipids and the APIs and to form a uniform gel.

Another hurdle in the production of phospholipid depots relates to the difficulty in preparing a sterile depot suitable for injection. Many drugs are heat-sensitive and cannot survive heat sterilization (e.g., autoclaving) or radiation sterilization. This is especially true for biological drugs such as metreleptin and pramlintide. In many cases, the only practical way to sterilize a protein-containing composition is by filtration through a 0.2- or 0.45-micron pore membrane to remove any microbial contaminants. With a 20-40% phospholipid content, the thick consistency of the gel compositions precludes any possibility of sterilization by filtration. Therefore, this invention also teaches unique methods for preparing gels that can be sterilized by filtration.

### BRIEF SUMMARY OF THE INVENTION

The present invention provides phospholipid-based depot gel compositions of at least one active pharmaceutical ingredient selected from pramlintide, a pramlintide analog, metreleptin, and a metreleptin analog (API) that are thixotropic and are injectable through fine needles. The present invention also provides methods for preparing sterile phospholipid-based depot gels that do not use any solvent that can damage the APIs, including metreleptin or pramlintide. Advantageously, the phospholipid-based depot gel (or “gel”) provides a prolonged circulation time in plasma for the at least one API following a subcutaneous injection and allows for no more than once-a-day injection.

As such, in one embodiment, the present invention provides a gel composition, comprising:

at least one active pharmaceutical ingredient selected from pramlintide, a pramlintide analog, metreleptin, and a metreleptin analog (API),

20 to 40% by weight of one or more phospholipids,

5 to 30% by weight of a medium chain triglyceride oil, and

10 to 56% by weight of water or a solvent,

wherein said gel composition is extrudable through a 25G ½ inch long needle from a 1 cc syringe at an extrusion rate of 2 cc/min by an applied force of no more than 90 Newtons.

The present invention also provides methods for preparing said gel compositions.

The present invention also provides a method for weight loss by injecting subcutaneously a gel composition as disclosed herein.

The present invention also provides a method for weight loss by injecting subcutaneously a gel composition as disclosed herein at a frequency such as once-daily, once-every 2 days, once-every 3 days, once-every 4 days, once-every 5 days, once-every 6 days, or once-a-week.

In certain embodiments, the gels of the current invention are thixotropic (FIG. 1), which is a desired property for good extrudability/injectability through a fine needle. In contrast, the same compositions, when prepared by known prior art methods, result in thick pastes that are very difficult or impossible to inject through a fine hypodermic needle.

These and other aspects, objects and embodiments will become more apparent when read with the accompanying detailed description and the figures that follow.

### BRIEF DESCRIPTION OF THE DRAWINGS

FIG. 1 shows the thixotropic property of F-210 (FIG. 1A) and F-211 (FIG. 1B) gel compositions according to EXAMPLE 3 and 4, respectively.

FIG. 2 illustrates prolonged plasma concentration-versus-time profiles of metreleptin and pramlintide in rats following a subcutaneous injection of the F-27 (FIGS. 2A and 2B), F-107 (FIGS. 2C and 2D) and F-207 (FIGS. 2E and 2F) gel compositions according to EXAMPLE 8, 7 and 1, respectively, in comparison to a solution formulation containing the same dose of metreleptin or pramlintide. The study details are given in EXAMPLE 9.

FIG. 3 shows a representative injection force versus time profile for the gel (F-207) according to EXAMPLE 1. The test measured the force (depicted on the Y-axis, negative values indicate pushing force) necessary to eject the gel from a 1 cc syringe through a 25G ½ inch long needle at rate of 2 cc/min over time (X-axis, in 1/10 sec). The maximum forces measured were about 12 Newtons (or about 2.5 pound-force).

FIG. 4 is a schematic representation of the speculated conversion from a fine emulsion (left) to a gel of this invention (right) upon removal of water.

### DETAILED DESCRIPTION OF THE INVENTION

### I. Definitions

An “active pharmaceutical ingredient” is a biologically active compound that has a therapeutic, prophylactic, or other beneficial pharmacological and/or physiological effect on a patient. In the gel compositions described herein, at least one active pharmaceutical ingredient selected from pramlintide, a pramlintide analog, metreleptin, and a metreleptin analog (API) is provided. Preferably, the gel compositions comprise pramlintide. In some embodiments, pramlintide and metreleptin are provided in the same gel composition.

The gel compositions generally comprise from about 0.01% (w/w) to about 50% (w/w) of the at least one API (based on the total weight of the composition). For example, the amount of API can be from about 0.1% (w/w) to about 30% (w/w) of the total weight of the composition. The amount of API will vary depending upon the desired effect, potency of the agent, the planned release levels, and the time span over which the drug will be released. In certain embodiments, the range of loading is between about 0.1% (w/w) to about 10% (w/w), for example, from about 0.1% (w/w) to about 5% (w/w), or from about 1% to about 5% (w/w).

When the API is pramlintide or a pramlintide analog, suitable release profiles can be obtained when the drug is loaded at about 0.1% (w/w) to about 0.5% (w/w), including at about 0.1% (w/w) to about 0.3% (w/w), at about 0.1% (w/w), at about 0.2% (w/w), at about 0.3% (w/w), and preferably, at about 0.28% (w/w) or at about 0.14% (w/w). When the API is metreleptin or a metreleptin analog, suitable release profiles can be obtained when the API is loaded at about 1.0% (w/w) to about 10.0% (w/w), including at about 1.0% (w/w) to about 5.0% (w/w), at about 1.0% (w/w), at about 2.0% (w/w), at about 3.0% (w/w), at about 4.0% (w/w), and at about 5.0% (w/w). Preferably, metreleptin or a metreleptin analog is loaded at about 2.0% (w/w) or about 4.0% (w/w).

### A. Metreleptin

The gel compositions disclosed herein include recombinant human methionyl leptin, also known as metreleptin, and metreleptin analogs. Metreleptin is an analog of human leptin, and has been studied as a potential treatment for obesity, type 2 diabetes, and lipodystrophy. Metreleptin has the following amino acid sequence (SEQ ID NO:1): MVPIQKVQDDTKTLIKTIVTRINDISHTQSVSSKQKVTGLDFIPGLHPILTLSKMDQ TLAVYQQILTSMPSRNVIQISNDLENLRDLLHVLAFSKSCHLPWASGLETLDSLGG VLEASGYSTEVVALSRLQGSLQDMLWQLDLSPGC.

Metreleptin analogs contemplated in the gel compositions of the invention include compounds having at least 80% sequence identity to SEQ ID NO:1 and having leptin activity. In some embodiments, the sequence identity is within the range 80%-100%. In some embodiments, the sequence identity is within the range 80%-90%. More preferably the analog sequence has at least 80%, 90%, or 95% amino acid sequence identity with the SEQ ID NO:1. The metreleptin analogs may also comprise conservative or non-conservative amino acid substitutions (including non-natural amino acids and L and D forms).

The term “leptin activity” includes leptin binding activity and leptin functional activity. The skilled artisan will recognize metreleptin analog compounds with leptin activity using suitable assays for measuring leptin binding or leptin functional activity. Metreleptin analog compounds can have an IC₅₀ of about 200 nM or less, about 100 nM or less, or about 50 nM or less, or about 5 nM or less, or about 1 nM or less, in a leptin binding assay, such as that described herein. The term “IC₅₀” refers in the customary sense to the half maximal inhibitory concentration of a compound inhibiting a biological or biochemical function. Accordingly, in the context of receptor binding studies, IC₅₀ refers to the concentration of a test compound which competes half of a known ligand from a specified receptor. Metreleptin analog compounds can have an EC₅₀ of about 20 nM or less, about 10 nM or less, about 5 nM or less, about 1 nM or less, or about 0.1 nM or less, in a leptin functional assay, such as that described herein. The term “EC₅₀” refers in the customary sense to the effective concentration of a compound which induces a response halfway between a baseline response and maximum response, as known in the art.

An exemplary leptin binding assay follows: leptin binding can be measured by the potency of a test compound in displacing ¹²⁵I-recombinant-Leptin (murine) from the surface membrane expressing chimeric Leptin (Hu)-EPO (Mu) receptor presented by the 32D OBECA cell line (J Biol Chem 1998; 273(29): 18365-18373). Purified cell membranes can be prepared by homogenization from harvested confluent cell cultures of 32D OBECA cells. Membranes can be incubated with ¹²⁵I-rec-Murine-Leptin and increasing concentrations of test compound for 3 hours at ambient temperature in 96-well polystyrene plates. Bound and unbound ligand fractions can then be separated by rapid filtration onto 96-well GF/B plates pre-blocked for at least 60′ in 0.5% PEI (polyethyleneimine). Glass fiber plates can then be dried, scintillant added, and CPM determined by reading on a multiwell scintillation counter capable of reading radiolabeled iodine.

An exemplary leptin functional assay follows: increased levels of phosphorylated STAT5 (Signal Transducer and Activator of Transcription 5) can be measured following treatment of 32D-Keptin cells ectopically expressing chimeric Hu-Leptin/Mu-EPO receptor with a test compound. The 32D-Keptin cells (identical to 32D-OBECA cells but maintained in culture with leptin) can be leptin weaned overnight and then treated with test compounds in 96-well plates for 30 minutes at 37° C. followed by cell extraction. The pSTAT5 levels in the cell lysates can be determined using the Perkin Elmer AlphaScreen® SureFire® pSTAT5 assay kit in a 384-well format (Proxiplate™ 384 Plus). The efficacy of test compounds can be determined relative to the maximal signal in cell lysates from cells treated with Human leptin.

### B. Pramlintide

The gel compositions disclosed herein include pramlintide and pramlintide analogs. Pramlintide is a synthetic hormone and an analog of human amylin. Pramlintide was approved by the FDA in March 2005, and is commercially available as an injectable drug sold under the brand name SYMLIN®. Pramlintide is used for lowering blood glucose levels to treat patients with type 1 and type 2 diabetes. Pramlintide is also reported to reduce body weight in animals and/or humans, and thus has been proposed for treating obesity and obesity-related disorders. Pramlintide has the following amino acid sequence (SEQ ID NO:2): KCNTATCATQRLANFLVHSSNNFGPILPPTNVGSNTY.

Pramlintide analogs contemplated in the gel compositions of the invention include compounds having at least 80% sequence identity to SEQ ID NO:2 and having amylin activity. In some embodiments, the sequence identity is within the range 80%-100%. In some embodiments, the sequence identity is within the range 80%-90%. More preferably the analog sequence has at least 80%, 90%, or 95% amino acid sequence identity with the SEQ ID NO:2. The pramlintide analogs may also comprise conservative or non-conservative amino acid substitutions (including non-natural amino acids and L and D forms).

The term “amylin activity” includes amylin receptor binding activity and amylin agonist activity. The skilled artisan will recognize pramlintide analog compounds with amylin activity using suitable amylin receptor binding assays or by measuring amylin agonist activity in, for example, soleus muscle assays. Pramlintide analog compounds can have an IC₅₀ of about 200 nM or less, about 100 nM or less, or about 50 nM or less, in an amylin receptor binding assay, such as that described herein, in U.S. Pat. No. 5,686,411, and US Publication No. 2008/0176804, the disclosures of which are incorporated by reference herein in their entireties and for all purposes. Pramlintide analog compounds can have an EC₅₀ of about 20 nM or less, about 15 nM or less, about 10 nM or less, or about 5 nM or less in a soleus muscle assay, such as that described in U.S. Pat. No. 5,686,411.

An exemplary amylin receptor binding assay follows: RNA membranes can be incubated with approximately 20 pM (final concentration) of ¹²⁵I-rat amylin (Bolton-Hunter labeled, PerkinElmer, Waltham, Mass.) and increasing concentrations of test compound for 1 hour at ambient temperature in, for example, 96-well polystyrene plates. Bound fractions of well contents can be collected onto a 96 well glass fiber plate (pre-blocked for at least 30 minutes in 0.5% PEI) and washed with 1×PBS using a Perkin Elmer plate harvester. Dried glass fiber plates can be combined with scintillant and counted on a multi-well Perkin Elmer scintillation counter.

The phrase “Acceptable Injectability Criterion” as used herein includes quantitatively defining a formulation that requires an applied force of no more than 90 Newtons (or 18 pounds force) to extrude the formulation from a 1 cc syringe through a 25G ½ inch long needle at rate of 2 cc/min. The “Acceptable Injectability Criterion” defines the maximum force acceptable for a typical subcutaneous injection.

The term “acidifying agent” includes a pharmaceutically acceptable acid such as hydrochloric acid, acetic acid, and sulfuric acid, and the like.

As used herein, the term “alkalizing agent” includes a pharmaceutically acceptable base such as sodium hydroxide, potassium hydroxide, ammonium hydroxide, lysine, arginine, and the like.

As used herein, the term “antimicrobial preservative or preservative” includes a pharmaceutical additive that can be added to an injectable pharmacologically active agent and be used to inhibit the growth of bacteria and fungi. The antimicrobial preservatives useful in this invention include, but are not limited to, cresols, phenol, benzyl alcohol, ethanol, chlorobutanol, parabens, imidura, benzylkonium chloride.

As used herein, the term “anhydrous” means substantially absent of water. For example, an anhydrous gel means that the water content in the gel is less than 2%, preferable less than 1% or more preferable less than 0.5%.

As used herein, the term “antioxidant” includes primarily reducing agents. The reducing agents useful in this invention include, but are not limited to, ascorbic acid or salts thereof, ascorbyl palmitate, sodium metabisulfite, propyl gallate, butylated hydroxyanisole, butylated hydroxytoluene, tocopherol, methionine or salts thereof, citric acid or salts thereof, reducing sugars, or mixtures thereof.

As used herein, the term “aqueous phase” includes a water solution containing pharmaceutically acceptable additives, such as acidifying, alkalizing, pH buffering, chelating, condensing and solubilizing agents, antioxidants and antimicrobial preservatives, tonicity/osmotic modifying agent, other biocompatible materials or therapeutic agents. In certain embodiments, such additives assist in stabilizing the pharmacologically active agent and depot compositions and in rendering the compositions biocompatible.

As used herein, the term “depot” refers to phospholipid-based gel composition that is capable of releasing at least one API in a slow or controlled manner into the surrounding tissues to achieve a prolonged duration of action, in comparison with an aqueous solution of the API. A depot composition may be administered by injection, instillation, or implantation into soft tissues, a certain body cavity or occasionally into a blood vessel with injection through fine needles being the preferred method of administration. A depot of the present invention is intended to provide (1) convenient or less frequent dosing, such as once-daily, once-every 2 days, once-every 3 days, once-every 4 days, once-every 5 days once-every 6 days or once-a-week, (2) prolonged action, (3) improved safety and/or (4) better drug efficacy. The term “depot” can be used interchangeably with “sustained-release,” “slow-release,” “timed-release,” “extended-release,” “delayed-release,” “long-acting,” or “controlled-release.”

As used herein, the term “emulsion” includes a mixture of immiscible oil phase and aqueous phase, where the oil phase comprises the oil and phospholipids and is in form of small droplets (the dispersed phase), which are suspended or dispersed in the aqueous phase (continuous phase). The “primary emulsion” formed in accordance with the present invention is typically optically opaque and possesses a finite stability. The “fine emulsion” formed in accordance with the present invention is typically translucent, having average droplet diameters of less than 200 nm, and filterable through a 0.2 micron filter.

As used herein, the term “a fine needle” or “fine hypodermic needle” includes a small-diameter, hollow needle that is used with a syringe to inject substances into the body. The outer diameter of the needle is indicated by the needle gauge system. According to the Stubs Needle Gauge system, hypodermic needles in common medical use range from 7 gauge (the largest) to 33 (the smallest). The word “fine,” as used herein, includes needles ranging from 21 to 33 gauge (G), preferably 25G to 31G and most preferably 25G to 29G. The definition for the fine hypodermic needle applies to both re-usable and disposable types. Disposable needles can be embedded in a plastic or aluminum hub that attaches to the syringe barrel by means of a press-fit or twist-on fitting or the “Luer Lock” connections or be permanently attached to the syringe barrel.

As used herein, the term “heat-sensitive” means that a given drug can lose 3% or more of its potency or concentration after autoclave treatment, for example at 121° C. for 15-20 min. Both metreleptin and pramlintide are heat-sensitive. For these drugs, terminal sterilization procedures that use heat (or autoclaving) are not feasible.

As used herein, the term “injectable or extrudable” includes meeting the Acceptable Injectability Criterion as previously defined above.

As used herein, the term “metal ion chelating agent or chelator” includes a metal ion chelator that is safe to use in an injectable product. A metal ion chelator works by binding to metal ions and thereby reduces the catalytic effect of metal ion on the oxidation, hydrolysis or other degradation reactions. Metal chelators that are useful in this invention may include disodium edetate (EDTA), glycine and citric acid and the respective salts thereof.

As used herein, the term “medium chain triglycerides or medium chain triglyceride oil” includes natural or synthetic triglycerides of fatty acids having 6 to 12 carbons. Medium chain triglycerides are represented by the compound of Formula (I):

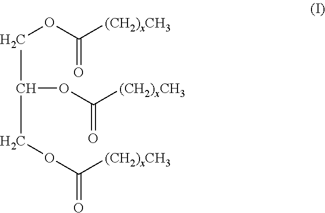

wherein each x is independently 4, 6, 8, or 10. When x is 4, the chain is referred to as a C₆ fatty acid. When x is 6, the chain is referred to as a C₈ fatty acid. When x is 8, the chain is referred to as a C₁₀ fatty acid. When x is 10, the chain is referred to as a C₁₂ fatty acid. In various embodiments, each x is the same integer; two x are the same integer and one x is a different integer; or each x is a different integer.

The skilled artisan will appreciate that a mixture of medium chain triglycerides may result from any process (e.g., fractionation, hydrogenation) used to prepare medium chain triglycerides. For example, substantially all of the medium chain triglycerides obtained from fractionated coconut oil may comprise C₈ and/or C₁₀ fatty acids; however, there may be some medium chain triglycerides containing C₆ and/or C₁₂ fatty acids.

A preferred medium chain triglyceride for this invention comprises 0 to 2 wt % C₆ fatty acid, 50 to 65 wt % C₈ fatty acid, 30 to 45 wt % C₁₀ fatty acid, and 0 to 2 wt % C₁₂ fatty acid, and which is commercially available as MIGLYOL® 812. The weight % is based on the total fatty acid content of the triglycerides.

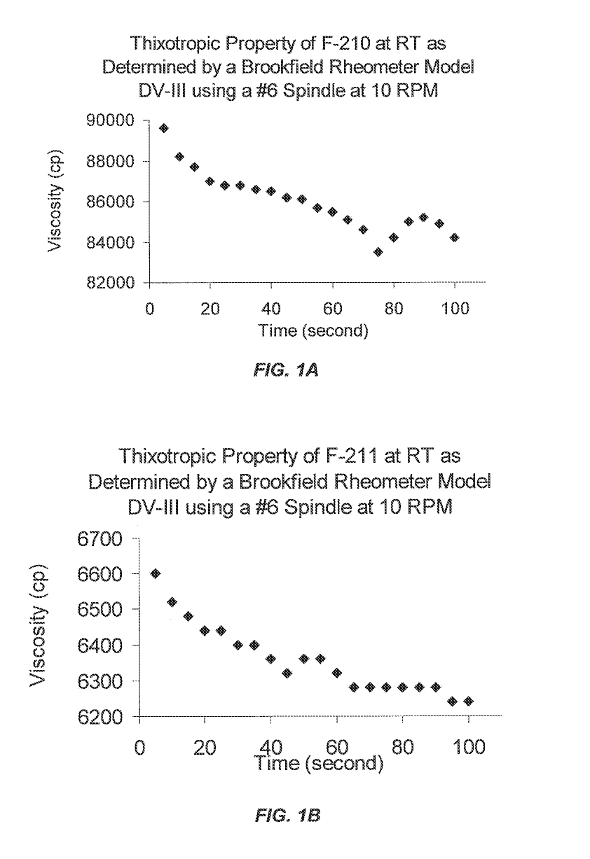

The term “Phospholipid-based gel,” or “gel” as used herein includes transparent, translucent or opaque semi-solid mass that comprises 20-40% phospholipids and meets the “Acceptable Injectability Criterion.” In certain preferred embodiments, the gels are thixotropic (FIG. 1).

As used herein, the term “pH buffering agent” includes a pharmaceutically acceptable pH buffer such as phosphate, acetate, citrate, bicarbonate, histidine, TRIS, and the like.

As used herein, the term “phospholipid” includes a class of lipids and are a major component of all cell membranes and contain a diglyceride, a phosphate group, and a simple organic molecule such as choline. A preferred phospholipid for this invention is a phosphotidylcholine. The more preferred phospholipid is 1-palmitoyl-2-oleoyl-sn-glycero-3-phosphocholine or POPC.

As used herein, the term “solvent” refers to non-aqueous liquids that are suitable and safe for subcutaneous injection. For example, a solvent can be propylene glycol, glycerol, sorbitol, polyethylene glycol, or mixtures thereof. The preferred solvent is glycerol or glycerin.

As used herein, the term “solubilizing agent” includes primarily surfactants such as polysorbate 80.

As used herein, the term “stabilizer” includes a pharmaceutically acceptable chemicals that (1) are capable of decrease solubility, (2) alters release rate, or (3) increases stability of the API. For example, zinc chloride forms insoluble crystals with metreleptin and causes the metreleptin to release slowly.

As used herein, a “sugar” includes a safe and biocompatible carbohydrate agent that protects the fine emulsion during drying by maintaining the discrete and sub-micron oil droplets. The sugars useful for this invention include monosaccharides, disaccharides, polysaccharides, poly-ols, dextrins, starches, celluloses and cellulose derivatives, or mixtures thereof. For instance, in certain embodiments, the sugar is mannitol, sorbitol, xylitol, lactose, fructose, xylose, sucrose, trehalose, mannose, maltose, dextrose, dextran, or a mixture thereof. In certain embodiments, the preferred sugar is sucrose.

As used herein, the term “thixotropic” refers to the property of certain gels that are thick (viscous) under normal conditions, but become thin or less viscous over time when sheared, shaken, or extruded (FIG. 1). A thixotropic gel exhibits a stable form at rest but becomes more injectable when subject to an extrusion force, resulting in good injectability.

### II. Embodiments

The present invention provides a thixotropic gel composition, comprising:

2 to 4% by weight of metreleptin

0.14 to 0.28% by weight of pramlintide

20 to 40% by weight of one or more phospholipids

5 to 10% by weight of a medium chain triglyceride oil, and

47 to 56% by weight of water

wherein said gel composition is extrudable through a 25G ½ inch long needle from a 1 cc syringe at an extrusion rate of 2 cc/min by an applied force of no more than 30 Newtons.

The present invention also provides an anhydrous gel composition, comprising:

2 to 4% by weight of metreleptin

0.14 to 0.28% by weight of pramlintide

20 to 40% by weight of one or more phospholipids

15 to 30% by weight of a medium chain triglyceride oil, and

25 to 35% by weight of glycerin

wherein said gel composition is extrudable through a 25G ½ inch long needle from a 1 cc syringe at an extrusion rate of 2 cc/min by an applied force of no more than 90 Newtons.

The present invention also provides a gel composition, comprising:

2 to 4% by weight of metreleptin

0.14 to 0.28% by weight of pramlintide

20 to 40% by weight of one or more phospholipids

5 to 10% by weight of a medium chain triglyceride oil, and

25 to 35% by weight of glycerin

wherein said gel composition is extrudable through a 25G ½ inch long needle from a 1 cc syringe at an extrusion rate of 2 cc/min by an applied force of no more than 90 Newtons.

In accordance with the practice of the present invention, the selection of a phospholipid for use in the depot compositions is determined by ability of the phospholipid to (1) form an oil-in-water emulsion and maintain the small droplet size through the manufacturing process and afterwards in storage, (2) be chemically compatible with the pharmacologically active agent and (3) provide the desired depot or sustained release properties for the pharmacologically active agent. Certain combinations of phospholipids can be utilized to form the depot such as POPC and DMPG Na. An optional phospholipid or phospholipid combination for a depot composition can be selected using the physical and chemical screening test methods known to those skilled in the art.

In another embodiment, the gel compositions of the present invention comprise 20-40% by weight, 22 to 35% by weight, and more preferably 24 to 30% by weight of a phospholipid such as 20, 21, 22, 23, 24, 25, 26, 27, 28, 29, 30, 31, 32, 33, 34, 35, 36, 37, 38, 39 or 40% by weight of a phospholipid or a mixture of phospholipids.

In one embodiment, a gel composition of the present invention comprises 10-60% water by weight.

In a yet another embodiment, the gel compositions of the present invention comprise medium chain triglyceride oil. The preferred concentration of medium chain triglyceride oil is 5 to 30%. An exemplary medium chain triglyceride oil is MIGLYOL® 812.

In a preferred embodiment, a sugar can be used in the present gel compositions. The preferred sugars are sucrose. The preferred concentration of sucrose is 0.5 to 20%, preferably 1 to 15% and more preferably 2 to 10%, such as 2%, 3%, 4%, 5%, 6%, 7%, 8%, 9% or 10% of the gel weight.

In one embodiment, the present invention provides gel compositions comprising pramlintide, satisfies the Acceptable Injectability Criterion, and are able to deliver pramlintide in sustained release profiles following an subcutaneous injection.

In one embodiment, the present invention provides gel compositions containing metreleptin and pramlintide, satisfies the Acceptable Injectability Criterion and are able to deliver metreleptin and pramlintide in sustained release profiles following an subcutaneous injection.

In one embodiment, the present invention provides methods to prepare gel compositions that are compatible with heat-sensitive metreleptin and pramlintide and permit sterilization by filtration of the emulsion intermediate through a 0.2 micron pore membrane, thus eliminating the need for an aseptic process or terminal sterilization using heat or radiation.

In another embodiment, the present invention provides methods to prepare gel such gel compositions, without the use of damaging amounts of organic solvent.

In certains embodiment, the invention gel compositions may contain a functional pharmaceutical excipient such as acidifying agents, alkalizing agents, pH buffering agents, metal ion chelators, antioxidants, stabilizers, preservatives, or a mixture thereof. The selection of a functional excipient(s) in a gel composition can be made based on stability requirement or other pharmaceutical considerations known by those skilled in the art.

In certain embodiments, the gel composition of the present invention maintains a plasma concentration of greater than 1 ng/mL for metreleptin and greater than 10 pg/mL for pramlintide 24 hours after an subcutaneous injection of 20 mg/kg metreleptin and 1.44 mg/kg pramlintide in rats.

In one embodiment, the present invention provides certain gel compositions that surprisingly satisfies or requires even less injection force than the Acceptable Injectability Criterion.

In another preferred embodiment, this invention relates to gel compositions, in their injectable, stable and sterilized form, that provide unique release profiles for metreleptin and pramlintide that are prolonged for at least 24 hours. Such release profile is highly desirable for metreleptin and pramlintide, which have short half-lives, and permit them to be maintained at the efficacious concentration levels in the circulation for a prolonged time.

In a preferred embodiment, the gel compositions is administered at once-a-day, once-every 2 days, once-every 3 days, once-every 4 days, once-every 5 days, once-every 6 days, once-every 7 days, once-every 10 days, once-every 14 days, or once-every 30 days.

### III. Methods of Making

Surprisingly, the gels prepared according to the methods of preparation of the present invention are easily injectable through fine needles. In some formats, the gels are partially translucent in appearance and silky smooth to the touch. In preferred embodiments, the gels are thixotropic, which are desired properties for good injectability through a fine needle.

In one embodiment, the present invention provides a method for preparing a gel composition, the method comprising:
a) combining all components including at least one active pharmaceutical ingredient selected from pramlintide, a pramlintide analog, metreleptin, and a metreleptin analog (API);
b) adding an excessive amount of water (more than what needed in the final composition);
c) mixing to form a primary emulsion;
d) adjusting pH to the targeted pH;
e) homogenizing the primary emulsion to form a fine emulsion with an average droplet size less than 200 nm in diameter;
f) passing the fine emulsion through a 0.2-micron filter; and
g) removing the excessive water to obtain the final gel composition.

In certain embodiments, the present invention provides a method for preparing a thixotropic gel composition, the method comprising:
a) combining all inactive components (i.e. without the API);
b) adding an excessive amount of water (more than what needed in the final composition);
c) mixing to form a primary emulsion;
d) adjusting pH to the targeted pH;
e) homogenizing the primary emulsion to form a fine emulsion with an average droplet size less than 200 nm in diameter;
f) passing the fine emulsion through a 0.2-micron filter;
g) adding the at least one API and mix well; and
h) removing the excessive water to obtain the final gel composition.

In a preferred embodiment, a high-shear, high-energy or high-pressure homogenizer (such as the microfluidizers from Microfluidics International Corporation) is used to convert the primary emulsion to the fine emulsion with average diameter less than 200 nm, preferable less than 100 nm and most preferably less than 50 nm. The reduction of droplets allows for the filtration of the fine emulsion through the 0.2-micron filter and greatly reduces viscosity and increases the injectability of the final gels.

After homogenization in a microfluidizer to reduce the droplet size to about 50 nm, the resulting fine emulsion is a clear, nearly transparent, and water-like liquid with a remarkably reduced viscosity. After removing the excessive water, the final gel satisfies the Acceptable Injectability Criterion. The fine emulsion can also be filtered through a 0.2-micron filter membrane, allowing sterilization of the gel preparations prior to parenteral administration. In contrast, the same phospholipid-containing composition without this homogenization step is not filterable through the same membranes.

Emulsions are thermodynamically unstable systems. If not processed properly, the emulsion droplets will aggregate, merge, grow in size and eventually result in the oil phase separating from the water phases (i.e., creaming out). When this happens the benefit of the reduced viscosity provided by the fine emulsion is lost. Surprisingly, in accordance with the practice of the present invention, the addition of certain sugars provides an unexpected protective effect for the fine emulsion against the aggregation of the droplets during the water removal processes. The presence of sugar in the fine emulsion thus keeps droplets essentially unchanged during the water removal step using the conditions disclosed herein. In contrast, a composition without sugar tends to be much less injectable.

In certain aspects, the gels of this invention can re-form the fine emulsion upon mixing in water, suggesting that the gels comprise discrete nanometer-sized droplets.

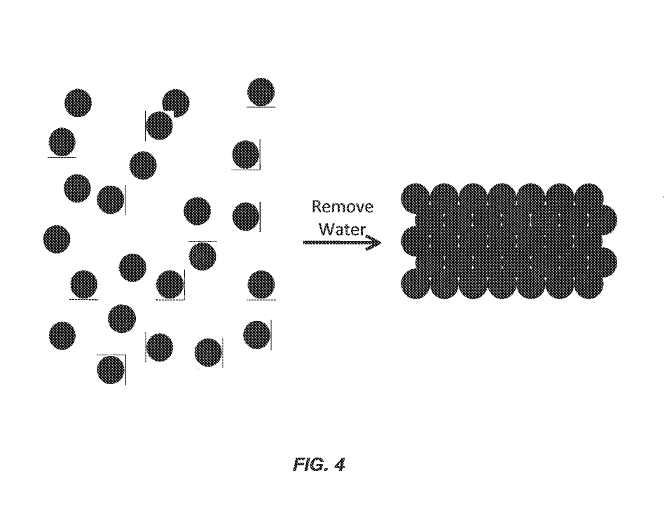

Not wishing to be bound by a theory or mechanism of the invention, it appears that the superior injectability offered by the gels of this invention is attributable to the extremely small droplets created by homogenization. This inventor speculates that by removing the water from the fine emulsion, the nanometer sized droplets stack together to form a certain organized structure like many small deformable “balloons” filled with oil and stacked together with water in the interstitial space. As the water is removed, the interstitial space is minimized causing the balloons to deform to compress into each other to form a more rigid structure i.e., a gel, but rather than fusing into each other, the balloons remain discrete in the gel phase. When an external force is applied (such as from a syringe plunger), the gel easily deforms and conforms to the needle bore because of the very small and discrete droplets, thus allowing for a superior injectability. FIG. 4 is a schematic representation of the speculated convention from a fine emulsion (left) to a gel of this invention (right) upon removal of water. The dark dots depict the nanosized droplets in the fine emulsion, and the space between the dots is filled with water with sugar. As the water or solvent is removed, the droplets become structurally organized into the gel.

In another embodiment, the filtration of the fine emulsion may be performed using a vacuum filtration method, centrifugation filtration, or pressurized filtration method. Various models or makes of 0.2-micron pore filter membranes are available. Examples include Sartopore, Sartobran P, Millipore, and the like. In some cases, a pre-filter with a larger pore size may be used. The primary reason for the filtration step is to sterilize the preparation.

In yet another embodiment, removal of water from the fine emulsion can be done by various drying methods, for example, by rotational vacuum drying method or by sweeping the nanodispersion with air or nitrogen gas (“air drying”). The rotational vacuum drying can be performed using commercially-available rotational evaporators such as a Rotavap (Buchi). The air drying is accomplished by mechanically stirring the nanodispersion while sweeping its surface with a stream of air or nitrogen gas. The air or nitrogen gas may be filtered through a 0.2-micron pore filter to sterilize first. Nitrogen gas is preferred if any components in the composition are prone to oxidation.

In some embodiments, the gels of this invention are filled into syringes to certain volume under aseptic conditions and are ready for injecting after attaching needles to the syringes. The pre-filled syringe format is convenient for self-administration. The preferred syringe size is 1-10 mL and the preferred needle size is 25-29G.

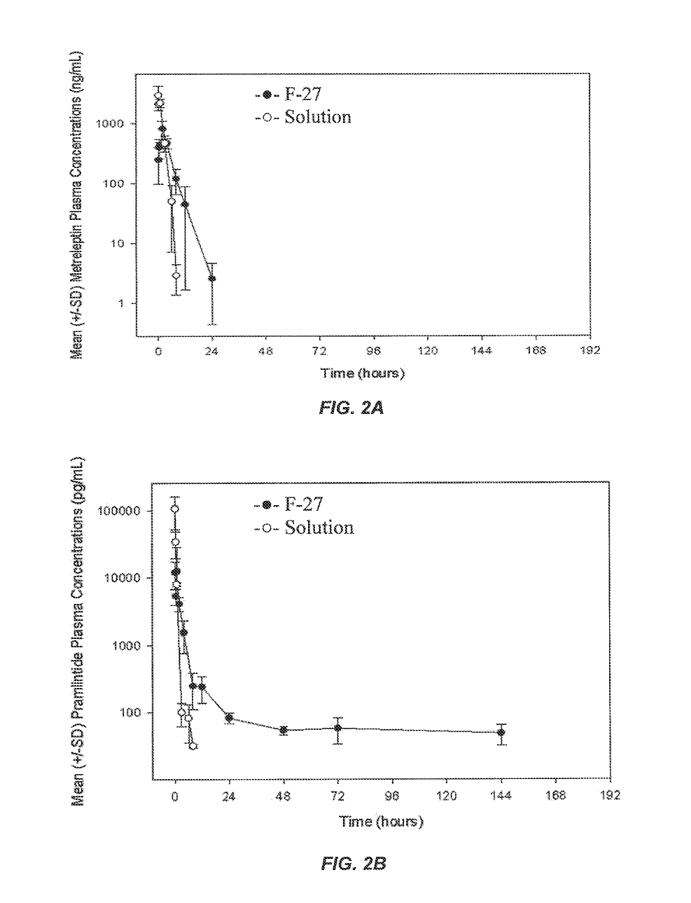

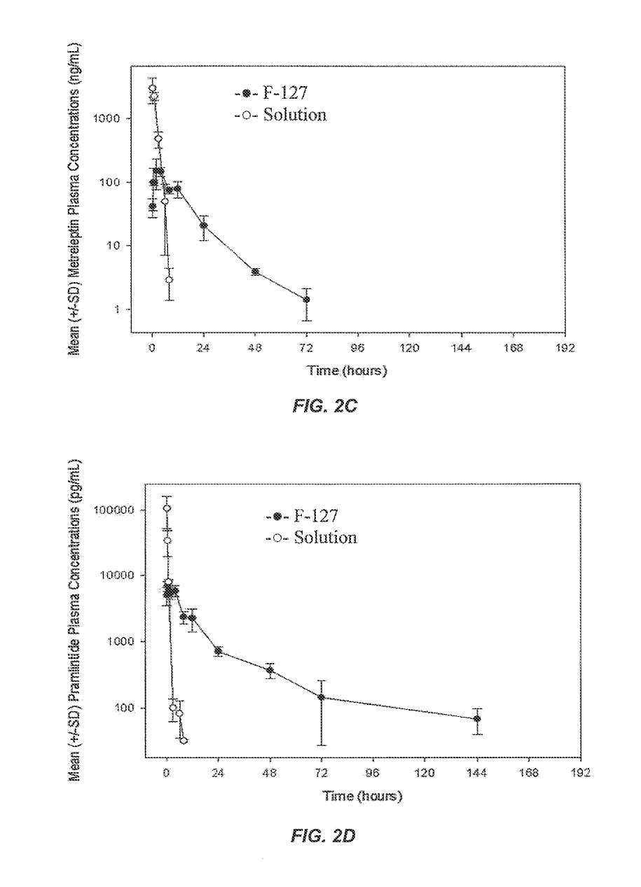

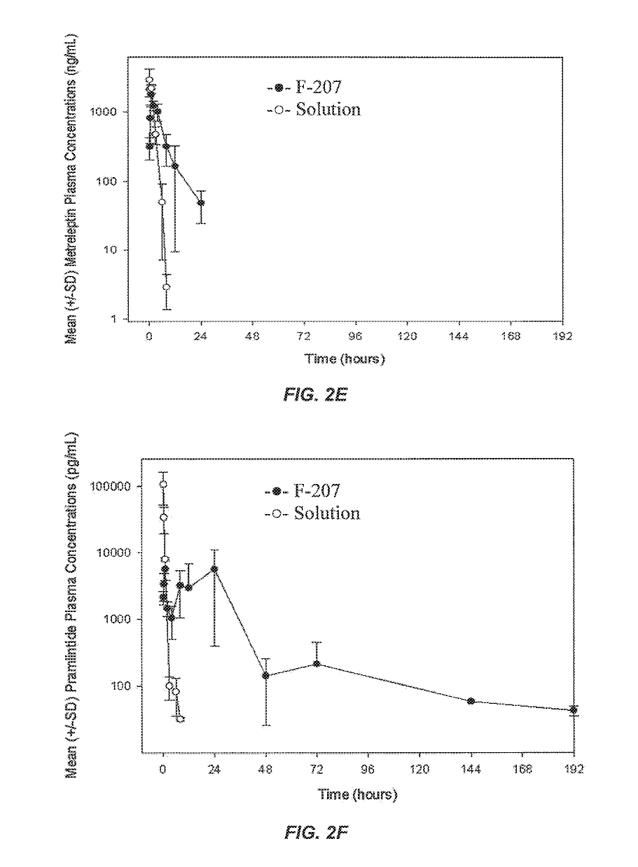

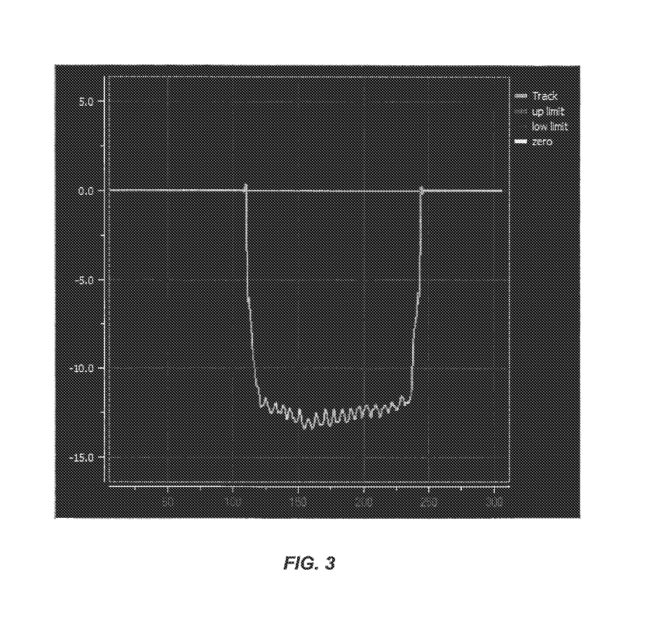

In one embodiment, the gels of this invention, after being injected into a soft tissue (e.g., subcutaneous or intramuscular injection), provide a slow drug release in vivo as shown by prolonged plasma concentration versus time profiles for both metreleptin and pramlintide, compared to the same doses of metreleptin and pramlintide given in a solution formulation (FIG. 2).

In certain aspects, a gel of this invention has a viscosity of about 100, 200, 500, 1000, 3000, and 5000 centipoise (cP). In certain aspects, the viscosity is at about 5000, 10,000, 50,000, 75,000, 1×10⁵, 1×10⁶, 1×10⁷, 1×10⁸ or 1×10⁹ cP at RT. In yet other aspects, the gel is thixotropic (FIG. 1).

In certain aspects, the gel formulation of the present invention is acidic to neutral. In certain aspects, the formulation has a pH between pH 2 and pH 8.5, preferably between pH 3 and pH 6, or more preferably between pH 4 and pH 5.

The invention will now be described in greater detail by reference to the following non-limiting examples.

### Example 1

### Preparation of a Gel Containing Metreleptin and Pramlintide (F-207)

F-207 Composition

| Component | % (wt) |
|---|---|
| Metreleptin* | 2.00 |
| Sodium glutamate* | 0.17 |
| Sucrose* | 1.02 |
| Glycine* | 2.04 |
| Polysorbate 20* | 0.01 |
| Pramlintide | 0.14 |
| 1-Palmitoyl-2-oleoyl-sn-glycero-3-phosphocholine (POPC) | 30.0 |
| Medium chain triglycerides (Miglyol 812) | 6.13 |
| Sucrose | 9.00 |
| Ethylenediaminetetraacetic acid, dehydrate, disodium (EDTA) | 0.20 |
| L-methinonine | 0.10 |
| Benzyl alcohol | 1.00 |
| Water for Injecttion (WFI) | 48.19 |
| Total | 100 |

*These components were carried into the formulation from the metreleptin stock solution used.

### Procedure

The F-207 gel was prepared as follows:
1. Weigh out all the components except WFI into a container.
2. Add WFI to 3.67 times the batch size weight.
3. Mix well using a high shear mixer to obtain a primary emulsion.
4. Homogenize the primary emulsion using a microfluidizer (Microfluidics International Corp Model M-110EH) to obtain a fine emulsion by reducing the average droplet size to less than 100 nm in diameter.
5. Adjust pH to 4.6+/−0.1 with HCl/NaOH.
6. Filter the fine emulsion through a 0.2-micron disposable vacuum filter (Nalgene) in a biosafety hood to sterilize the emulsion.
7. Aseptically, remove water by blowing with 0.2 μm-filtered nitrogen gas NF into the emulsion with stirring until the water content reaches final water content in the gel (i.e. 48.19%).
8. Aseptically, mix well the gel to uniformity.
9. Aseptically, centrifuge gel to remove air bubbles.
10. Aseptically, fill 0.2-0.4 mL gel into each sterile syringe (e.g. BD 1 mL syringe Luer-lok Tip).
11. Aseptically seal the syringes with Luer-lock caps (Qosina Non-vented FLL Cap w/Internal Pin, P/N 17542).
12. Store the syringes at 2-8° C.

F-207 was a smooth and opaque gel. The metreleptin and pramlintide concentrations were confirmed by an RP-HPLC analysis.

The injectability of F-207 was determined against the Acceptable Injectability Criterion. The maximum force required during the injectability test is recorded as the most relevant measurement parameter for injectability. For the injectability test, 0.5 mL F-207 was filled into a 1cc B-D syringe (B-D Luer-Lok Tip, ref 309628) to which a ½″ long 25G needle (EXEL, Hypodermic needle, ref 26403) was attached. The filled syringe was loaded onto a syringe pump to which a force meter (Advanced Precision Instrument Model HP-500) was attached against the plunger end to measure to force applied to extrude the syringe contents. The syringe pump was set at 2 cc/min speed and 0.4 mL extrusion volume. The force was recorded in Newtons. In the “push” mode, the force is recorded as negative. With a maximal injection force of about 12 Newtons (average of 2 tests), F-207 is regarded as highly injectable and meeting the Acceptable Injectability Criterion.

### Example 2

### Preparation of a Gel Containing Metreleptin & Pramlintide (F-209)

F-209 in the following composition was prepared using the same method as described in Example 1. For F-209, a new lot of metreleptin was used. The new lot was provided in a new stock solution that does not contain sucrose, glycine or polysorbate.

F-209 Composition

| Component | % (wt) |
|---|---|
| Metreleptin | 2.00 |
| Sodium glutamate | 0.17 |
| Pramlintide | 0.14 |
| 1-Palmitoyl-2-oleoyl-sn-glycero-3-phosphocholine (POPC) | 30.0 |
| Medium chain triglycerides (Miglyol 812) | 6.13 |
| Sucrose | 9.00 |
| Ethylenediaminetetraacetic acid, dehydrate, disodium (EDTA) | 0.20 |
| L-methinonine | 0.10 |
| Benzyl alcohol | 1.00 |
| Water for Injection (WFI) | 51.26 |
| Total | 100 |

F-209 was a smooth and opaque gel. The metreleptin and pramlintide concentrations were confirmed by an RP-HPLC analysis. Using the same method of Injectability test as described in Example 1, F-209 had a maximal injection force of 8.1 Newtons (average of 2 tests). Therefore, F-209 is regarded as highly injectable and meeting the Acceptable Injectability Criterion.

### Example 3

### Preparation of a Gel Containing Higher Concentrations of Metreleptin & Pramlintide (F-210)

F-210 in the following composition was prepared using the same method as described in Example 1. F-210 contains also 30% POPC but 2× metreleptin and pramlintide as compared to the Example 1 composition.

F-210 Composition

| Component | % (wt) |
|---|---|
| Metreleptin | 4.00 |
| Sodium glutamate | 0.34 |
| Pramlintide | 0.28 |
| 1-Palmitoyl-2-oleoyl-sn-glycero-3-phosphocholine (POPC) | 30.0 |
| Medium chain triglycerides (Miglyol 812) | 6.13 |
| Sucrose | 9.00 |
| Ethylenediaminetetraacetic acid, dehydrate, disodium (EDTA) | 0.20 |
| L-methinonine | 0.10 |
| Benzyl alcohol | 1.00 |
| Water for Injection (WFI) | 48.95 |
| Total | 100 |

F-210 was a smooth and opaque gel. The metreleptin and pramlintide concentrations were confirmed by an RP-HPLC analysis. Using the same method of Injectability test as described in Example 1, F-210 had a maximal injection force of 13.4 Newtons (average of 2 tests). Therefore, F-210 is regarded as highly injectable and meeting the Acceptable Injectability Criterion.

### Example 4

### Preparation of a Gel Containing Metreleptin & Pramlintide (F-211)

F-211 in the following composition was prepared using the same method as described in Example 1. F-211 also contains the same concentrations of metreleptin and pramlintide as in F-210 but with reduced POPC (24%).

F-211 Composition

| Component | % (wt) |
|---|---|
| Metreleptin | 4.00 |
| Sodium glutamate | 0.34 |
| Pramlintide | 0.28 |
| 1-Palmitoyl-2-oleoyl-sn-glycero-3-phosphocholine (POPC) | 24.0 |
| Medium chain triglycerides (Miglyol 812) | 4.90 |
| Sucrose | 9.00 |
| Ethylenediaminetetraacetic acid, dehydrate, disodium (EDTA) | 0.20 |
| L-methinonine | 0.10 |
| Benzyl alcohol | 1.00 |
| Water for Injection (WFI) | 56.18 |
| Total | 100 |

F-211 was a smooth and opaque gel. The metreleptin and pramlintide concentrations were confirmed by an RP-HPLC analysis. Using the same method of Injectability test as described in Example 1, F-211 had a maximal injection force of 8.6 Newtons (average of 2 tests). Therefore, F-211 is regarded as highly injectable and meeting the Acceptable Injectability Criterion.

### Example 5

### Preparation of a Gel Containing Metreleptin & Pramlintide with ZnCl₂ (F-216)

F-216 contains the same concentrations of metreleptin and pramlintide as F-210 but with reduced POPC (28%). In addition, zinc chloride was added to stabilize the preparation.

F-216 Composition

| Component | % (wt) |
|---|---|
| Metreleptin | 4.00 |
| Sodium glutamate | 0.34 |
| Pramlintide | 0.28 |
| ZnCl₂ | 3.00 |
| 1-Palmitoyl-2-oleoyl-sn-glycero-3-phosphocholine (POPC) | 28.0 |
| Medium chain triglycerides (Miglyol 812) | 6.13 |
| Sucrose | 9.00 |
| Ethylenediaminetetraacetic acid, dehydrate, disodium (EDTA) | 0.20 |
| L-methinonine | 0.10 |
| Benzyl alcohol | 1.00 |
| Water for Injection (WFI) | 47.95 |
| Total | 100 |

F-216 was prepared as follow:
(1) Dissolve pramlintide in the metreleptin stock solution which contains sodium glutamate.
(2) Pass the solution through a 0.2 μm sterile filters to sterilize.
(3) Dissolve ZnCl₂ in DI-water. Pass the solution through a 0.2 μm sterile filters to sterilize.
(4) Aseptically mix the filtered metreleptin and pramlintide solution with the filtered ZnCl₂ solution with stirring at room temperature for 30 min.
(5) Prepare a sterile fine emulsion vehicle (without metreleptin, pramlintide and ZnCl₂) using the same similar method as described in Example 1.
(6) Aseptically combine fine emulsion vehicle and the mixture prepared at Step 4.
(7) Aseptically, remove water by blowing with 0.2 μm-filtered nitrogen gas NF into the emulsion with stirring until the water content reaches final water content in the gel (i.e. 47.59%).
(8) Aseptically, mix well the gel to uniformity.
(9) Aseptically, centrifuge gel to remove air bubbles.
(10) Aseptically, fill 0.2-0.4 mL gel into each sterile syringe (e.g. BD 1 mL syringe Luer-lok Tip).
(11) Aseptically seal the syringes with Luer-lock caps (Qosina Non-vented FLL Cap w/Internal Pin, P/N 17542).
(12) Store the syringes at 2-8° C.

F-216 was a smooth and white gel. The metreleptin and pramlintide concentrations were confirmed by an RP-HPLC analysis. Using the same method of Injectability test as described in Example 1, F-216 had a maximal injection force of 19.2 Newtons (average of 2 tests). Therefore, F-216 is regarded as highly injectable and meeting the Acceptable Injectability Criterion.

### Example 6

### Preparation of Gel Containing Metreleptin & Pramlintide with DMPG-Na (F-217)

F-217 contains the same concentrations of metreleptin and pramlintide as F-216 but with reduced POPC (26%). In addition, DMPG-Na was added to stabilize the preparation. F-217 was prepared using the same method as described in Example 5.

F-217 Composition

| Component | % (wt) |
|---|---|
| Metreleptin | 4.00 |
| Sodium glutamate | 0.34 |
| Pramlintide | 0.28 |
| DMPG-Na | 2.00 |
| 1-Palmitoyl-2-oleoyl-sn-glycero-3-phosphocholine (POPC) | 26.0 |
| Medium chain triglycerides (Miglyol 812) | 6.13 |
| Sucrose | 9.00 |
| Ethylenediaminetetraacetic acid, dehydrate, disodium (EDTA) | 0.20 |
| L-methinonine | 0.10 |
| Benzyl alcohol | 1.00 |
| Water for Injection (WFI) | 50.95 |
| Total | 100 |

F-217 was a smooth and white gel. The metreleptin and pramlintide concentrations were confirmed by an RP-HPLC analysis. Using the same method of Injectability test as described in Example 1, F-217 had a maximal injection force of 20.1 Newtons (average of 2 tests). Therefore, F-217 is regarded as injectable and meeting the Acceptable Injectability Criterion.

### Example 7

### Preparation of an Anhydrous Gel Containing Metreleptin & Pramlintide (F-127)

F-127 Composition

| Component | % (wt) |
|---|---|
| Metreleptin* | 2.00 |
| Sodium glutamate* | 0.17 |
| Sucrose* | 1.02 |
| Glycine* | 2.04 |
| Polysorbate 20* | 0.01 |
| Pramlintide | 0.14 |
| 1-Palmitoyl-2-oleoyl-sn-glycero-3-phosphocholine (POPC) | 22.0 |
| 1,2-Dimyristoyl-sn-glycero-3-phosphoglycerol, | 10.0 |
| sodium salt (DMPG-Na) | |
| Medium chain triglycerides (Miglyol 812) | 21.92 |
| Glycerin | 32 |
| Ethylenediaminetetraacetic acid, dehydrate, disodium (EDTA) | 0.20 |
| L-methinonine | 0.10 |
| Benzyl alcohol | 1.00 |
| Propylene glycol | 1.40 |
| Dehydrate alcohol | 6.00 |
| Total | 100 |

*These components were carried into the formulation from the metroleptin stock solution used.

### Procedure

The F-127 gel was prepared as follow:
1. Weigh out the required amounts of POPC, DMPG-Na, Medium chain triglycerides, glycerin, metreleptin stock solution, pramlintide, EDTA, L-methionine and benzyl alcohol into a container.
2. Add WFI to 4 times the batch size weight.
3. Mix well using a high shear mixer to obtain a primary emulsion.
4. Homogenize the primary emulsion using a microfluidizer (Microfluidics International Corp Model M-110EH) to obtain a fine emulsion by reducing the average oil droplet size to less than 100 nm in diameter.
5. Adjust pH to 4.6+/−0.1 with HCl/NaOH.
6. Aseptically, filter the fine emulsion through a 0.2-micron filter (Nalgene) in a biosafety hood to sterilize the emulsion.
7. Aseptically, remove water by freeze-drying the filtered emulsion until water content was less than 1% to obtain paste.
8. Aseptically, add the required sterile dehydrate alcohol and sterile propylene glycol into the paste to obtain a gel.
9. Aseptically, mix well the gel to uniformity.
10. Aseptically, centrifuge gel to remove air bubbles.
11. Aseptically, fill gel into sterile syringe (e.g. BD 1 mL syringe Luer-lok Tip).
12. Aseptically seal the syringes with Luer-lock caps (Qosina Non-vented FLL Cap w/Internal Pin, P/N 17542).
13. Store the syringes at 2-8° C.

F-127 was an opaque gel. The metreleptin and pramlintide concentrations were confirmed by an RP-HPLC analysis. Using the same method of Injectability test as described in Example 1, F-127 had a maximal injection force of 78 Newtons.

### Example 8

### Preparation of a Gel Composition Containing Metreleptin, Pramlintide & Glycerin (F-27)

F-27 Composition

| Component | % (wt) |
|---|---|
| Metreleptin* | 2.00 |
| Sodium glutamate* | 0.17 |
| Sucrose* | 1.02 |
| Glycine* | 2.04 |
| Polysorbate 20* | 0.01 |
| Pramlintide | 0.14 |
| 1-Palmitoyl-2-oleoyl-sn-glycero-3-phosphocholine (POPC) | 30.00 |
| Medium chain triglycerides (Miglyol 812) | 9.32 |
| Glycerin | 32.00 |
| Ethylenediaminetetraacetic acid, dehydrate, disodium (EDTA) | 0.20 |
| L-methinonine | 0.10 |
| Benzyl alcohol | 1.00 |
| Dehydrate alcohol | 6.00 |
| Water for Injection (WFI) | 16.00 |
| Total | 100 |

*These components were carried into the formulation from the metroleptin stock solution used.

F-27 was prepared using the similar method as described in Example 7 (where WFI was added with the dehydrated alcohol at Step 8). F-27 was a slightly translucent gel. The metreleptin and pramlintide concentrations were confirmed by an RP-HPLC analysis. Using the same method of Injectability test as described in Example 1, F-27 had a maximal injection force of 71 Newtons.

### Example 9

### Prolonged Pharmacokinetic Profiles Delivered by F-27, F-127 and F-0207 Following Subcutaneous Injections in Rats

This study was conducted to evaluate F-27 (a gel containing glycerin as described in EXAMPLE 8), F-127 (an anhydrous gel as in EXAMPLE 7) and F-207 (as in EXAMPLE 1) in rats by comparing their blood concentration versus time profiles, i.e. pharmacokinetic profiles with a reference formulations in which metreleptin and pramlintide were dissolved in an aqueous solution at the same doses.

Rats were placed into treatment groups (9 in each group). Each formulation was administered subcutaneously at 20 mg/kg dose for metreleptin and 1.44 mg/kg for pramlintide. Blood samples were taken at pre, 12, 24, 48, 72, 144, and 192 hr post-administration from the lateral tail vein. The concentrations of metreleptin and pramlintide in plasma were measured by an immunoenzymetric assay method.

All three gel compositions exhibited prolonged plasma-versus-time profiles for both metreleptin and pramlintide compared to the solution formulation given at the same doses (FIG. 2).

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group. Other embodiments are set forth within the following claims.

1. A gel composition, comprising:
at least one active pharmaceutical ingredient selected from pramlintide, a pramlintide analog, metreleptin, and a metreleptin analog (API),
20 to 40% by weight of one or more phospholipids,
5 to 30% by weight of a medium chain triglyceride oil, and
10 to 56% by weight of water or a solvent.

2. The gel composition of claim 1, wherein the gel is extrudable through a 25G ½ inch long needle from a 1 cc syringe at an extrusion rate of 2 cc/min by an applied force of no more than 30 Newtons.

3. The gel composition of claim 1, wherein the ingredient is pramlintide or a pramlintide analog.

4. The gel composition of claim 1, wherein the ingredient is metreleptin or a metreleptin analog.

5. The gel composition of claim 1, wherein the ingredient is pramlintide and metreleptin.

6. The gel composition of claim 5, wherein the gel maintains a plasma concentration above 1 ng/mL for metreleptin and above 10 picogram/mL for pramlintide within 24 hours following a subcutaneous injection of 20 mg/kg metreleptin and 1.44 mg/kg pramlintide in rats.

7. The gel composition of claim 5, wherein the metreleptin is in a concentration range of about 2 to about 4% and pramlintide in a concentration range of about 0.14 to about 0.28% by weight of the gel.

8. The gel composition of claim 1, wherein the solvent is glycerin.

9. The gel composition of claim 1, wherein the gel is anhydrous.

10. The gel composition of claim 1, wherein the phospholipid is POPC.

11. The gel composition of claim 1, wherein the medium chain triglyceride oil is Miglyol 812.

12. The gel composition of claim 1, wherein the gel further comprises a stabilizer selected from sucrose, glutamate, EDTA, methionine, polysorbate, zinc chloride, or a combination thereof.

13. The gel composition of claim 1, wherein the gel further comprises a preservative.

14. The gel composition of claim 13, wherein the preservative is selected from the group consisting of phenol, cresol, paraben, benzyl alcohol, chlorobutanol, thimerosol or a combination thereof.

15. A method for preparing a gel composition comprising,
at least one active pharmaceutical ingredient selected from pramlintide, a pramlintide analog, metreleptin, and a metreleptin analog (API),
20 to 40% by weight of one or more phospholipids,
5 to 22% by weight of a medium chain triglyceride oil, and
10 to 56% by weight of a solvent
wherein said gel composition is extrudable through a 25G ½ inch long needle from a 1 cc syringe at an extrusion rate of 2 cc/min by an applied force of no more than 90 Newtons, the method comprising:
Step 1: forming a primary emulsion comprising one or more phospholipid(s), medium chain triglyceride oil, a stabilizer and an excessive amount of water;
Step 2: homogenizing the primary emulsion to form a fine emulsion with an average droplet size between about 30 nm to about 200 nm in diameter;
Step 3: passing the fine emulsion through a 0.2-micron filter; and
Step 4: removing the excessive water to obtain a gel composition,
wherein the least one API is added to the composition at Step 1, 2, 3 or 4.

16. (canceled)

17. A method for the treatment of a human or non-human mammalian subject comprising administering to said subject a gel composition as claimed in claim 1.

18. The method of claim 17 for the treatment of a human or non-human mammalian subject in need thereof to combat at least one condition selected from diabetes, type I diabetes, type II diabetes, excess bodyweight, need for bodyweight reduction, obesity, hypertension and lipodystrophy.

19. A gel composition selected from F-207, F-209, F-210, F-211, F-216, and F-217.


SEQUENCE LISTING (2 sequences)

SEQ ID NO: 1 - protein, 147 aa, Homo sapiens
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 2 - protein, 37 aa, Artificial Sequence
  Description of Artificial Sequence Synthetic polypeptide
KCNTATCATQRLANFLVHSSNNFGPILPPTNVGSNTY